(19) United States
(12) Patent Application Publication
(10) Pub. No.: US 2008/0063667 A1
(43) Pub. Date: Mar. 13, 2008

(54) ProDer P 1 Expressed in a Prokaryotic Cell

(76) Inventors: Alain Jacquet, Gosselies (BE); Christel Matteotti, Gosselies (BE); David Walgraffe, Gosselies (BE)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/770,967

(22) Filed: Jun. 29, 2007

(30) Foreign Application Priority Data
Apr. 12, 2006 (GB) 0607377.9
Apr. 12, 2007 (EP) PCT/EP07/53554

(51) Int. Cl. A61K 39/00; A61P 43/00; C12P 21/04

(52) U.S. Cl. 424/275.1; 435/69.1

(57) ABSTRACT

This invention relates to novel methods for producing recombinant allergens, including but not limited to proDer p 1, the precursor form of the major protein allergen from Dermatophagoides pteronyssinus Der p 1, and to recombinant allergens produced by such methods. The invention further relates to the use of said recombinant allergens in formulating immunogenic compositions and vaccines effective in the prevention and/or the reduction of allergic responses to specific allergens.

This application claims priority to International Application No. PCT/EP2007/053554 filed Apr. 12, 2007, which claims the benefit of GB application No. 0607377.9 filed Apr. 12, 2006.

### FIELD OF THE INVENTION

This invention relates to novel methods for producing recombinant allergens, including but not limited to proDer p 1, the precursor form of the major protein allergen from Dermatophagoides pteronyssinus Der p 1, and to recombinant allergens produced by such methods. The invention further relates to the use of said recombinant allergens in formulating immunogenic compositions and vaccines effective in the prevention and/or the reduction of allergic responses to specific allergens.

### BACKGROUND OF THE INVENTION

Allergic responses in humans are common, and may be triggered by a variety of allergens. Allergic individuals are sensitized to allergens, and are characterized by the presence of high levels of allergen specific IgE in the serum, and possess allergen specific T-cell populations which produce Th2-type cytokines (IL-4, IL-5, and IL-13). Binding of IgE, in the presence of allergen, to FcεRI receptors present on the surface of mastocytes and basophils, leads to the rapid degranulation of the cells and the subsequent release of histamine, and other preformed and neoformed mediators of the inflammatory reaction. In addition to this, the stimulation of the T-cell recall response results in the production of IL-4 and IL-13, together cooperating to switch B-cell responses further towards allergen specific IgE production. For details of the generation of early and late phase allergic responses see Joost Van Neeven et al., 1996, Immunology Today, 17, 526. In non-allergic individuals, the immune response to the same antigens may additionally include Th1-type cytokines such as IFN-γ. These cytokines may prevent the onset of allergic responses by the inhibition of high levels of Th2-type immune responses, including high levels of allergen specific IgE. Importantly in this respect, is the fact that IgE synthesis may be controlled by an inhibitory feedback mechanism mediated by the binding of IgE/allergen complexes to the CD23 (FcεRII) receptor on B-cells (Luo et al., J. Immunol., 1991, 146(7), 2122-9; Yu et al., 1994, Nature, 369(6483): 753-6). In systems that lack cellular bound CD23, this inhibition of IgE synthesis does not occur.

Type I allergic diseases mediated by IgE against allergens such as bronchial asthma, atopic dermatitis and perrenial rhinitis affect more than 20% of the world's population. Current strategies in the treatment of such allergic responses include means to prevent the symptomatic effects of histamine release by anti-histamine treatments and/or local administration of anti-inflammatory corticosteroids. Other strategies which are under development include those which use the host's immune system to prevent the degranulation of the mast cells (Stanworth et al., EP 0 477 231 B1). Other forms of immunotherapy have been described (Hoyne et al., J. Exp. Med., 1993, 178, 1783-1788; Holt et al., Lancet, 1994, 344, 456-458).

While immediate as well as late symptoms can be ameliorated by pharmacological treatment, allergen-specific immunotherapy is the only curative approach to type I allergy. However, some problems related to this method remain to be solved. First, immunotherapy is currently performed with total allergen extracts which can be heterogeneous from batch to batch. Moreover, these allergen mixtures are not designed for an individual patient's profile and may contain unwanted toxic proteins. Second, the administration of native allergens at high doses can cause severe anaphylactic reactions and therefore the optimally efficient high dose of allergen for successful immunotherapy can often not be reached. The first problem has been addressed through alternative vaccination with better characterised and more reproducible recombinant allergens as compared to allergen extracts. The second problem, namely the risk of anaphylactic reactions induced by repeated injections of allergen extracts, can be minimised through the use of recombinant “hypoallergens”, whose IgE reactivity was altered by deletions or mutagenesis (Akdis, C A and Blaser, K, Regulation of specific immune responses by chemical and structural modifications of allergens, Int. Arch. Allergy Immunol., 2000, 121, 261-269).

Formulations have been described for the treatment and prophylaxis of allergy, which provide means to down-regulate the production of IgE, as well as modifying the cell mediated response to the allergen, through a shift from a Th2 type to a Th1 type of response (as measured by the reduction of ratio of IL-4:IFN-γ producing Der p 1 specific T-cells, or alternatively a reduction of the IL-5:IFN-γ ratio). This may for example be achieved through the use of recombinant allergens such as recDer p 1 with reduced enzymatic activity as described in WO 99/25823. However the immunogenicity of these recombinant allergens is thought to be similar to that of wild-type ProDer p 1 in terms of IgE synthesis induction.

Non-anaphylactic forms of allergens with reduced IgE-binding activity have been reported. Allergen engineering has allowed a reduction of IgE-binding capacities of the allergen proteins by site-directed mutagenesis of amino acid residues or deletions of certain amino acid sequences. At the same time, T-cell activating capacity is still conserved as T cell epitopes are maintained. This has been shown using several approaches for different allergens although with variable results. Examples have been published for the timothy grass pollen allergen Phl p 5b (Schramm G et al., 1999, J Immunol., 162, 2406-14), for the major house dust mite allergens Derf2 (Takai et al. 2000, Eur. J. Biochem., 267, 6650-6656), DerP2 (Smith & Chapman 1996, Mol. Immunol. 33, 399-405) and Derf1 (Takahashi K et al. 2001, Int Arch Allergy Immunol. 124, 454-60). One study has reported the generation of Derf1 hypoallergens by introductions of point mutations at the level of cysteine residues involved in disulfides bridges (Takahashi K Int Arch Allergy Immunol. 2001; 124(4): 454-60., Takai T, Yasuhara T, Yokota T, Okumura Y). However, if wild-type ProDerf1 was successfully secreted by P. pastoris, cysteine mutants concerning intramolecular disulfide bonds were, by contrast, not secreted.

Der p 1 Allergen

Allergens from the house dust mite Dermatophagoides pteronyssinus are one of the major causative factors associated with allergic hypersensitivity reactions. The group 1 allergen of Dermatophagoides pteronyssinus, Der p 1, is a major allergen, binding IgE in 80-100% of dust mite allergic sera (Chapman, M. D., et al. (1983). J. Allergy Clin. Immunol., 72: 27-33; Krillis, S., et al. (1984). J. Allergy Clin. Immunol., 74: 132-41). Der p 1 is produced in the mid-gut of the mite, where its role is probably related to the digestion of food. Up to 0.2 ng of proteolytically active Der p 1 is incorporated into each fecal pellet, each around 10-40 μm in diameter and, therefore, easily inspired into the human respiratory tract. This protein is frequently found in high concentrations in house dust: from 100 to 10000 ng/g of dust (Platts-Mills and Chapman (1987). J.Allergy Clin. Immunol., 80: 755-75; Wahn, U., et al. (1997). J. Allergy Clin. Immunol., 99: 763-69), but Der p 1 is thought to be associated with a range of particles and not just faecal material (DeLuca, et al. (1999). J. Allergy Clin. Immunol., 103: 174-75). Levels of 100 ng are associated with sensitization and the risk increases with increasing doses.

The cDNA coding for Der p 1 has been cloned and sequenced (Chua, K., et al. (1988). J. Exp. Med., 167: 175-82; Thomas, et al. (1988). Int. Arch. Allergy Appl. Immunol., 85: 127-29; Chua, K., et al (1993). Int. Arch. Allergy Immunol., 101: 364-8). Der p 1 is known to contain 222 amino acid residues in the mature protein and has a calculated molecular weight of 25 KDa. The Der p 1 encoding cDNA sequence reveals that, like many mammalian and plant proteinases, Der p 1 is synthesized in a precursor form of 320 amino acid residues, including a 18-amino acid signal peptide and 80-amino acid N-terminal prosequence. The maturation process of ProDer p 1 is not known, but it is thought that the enzyme is activated by proteolytic removal of the pro region or via autocatalytic processing. Overnight storage of purified Der p 1 preparations at room temperature results in almost complete loss of enzymatic activity due to autoproteolytic degradation (Machado et al., 1996, Eur.J.Immunol. 26, 2972-2980).

The Der p 1 sequence displays 30% homology with that of papain, the cysteine proteinase archetype (Robinson, C., et al. (1997). Clin. Exp. Allergy, 27 (1): 10-21) and shares more particularly homology in the enzymatically active regions, most notably the Cys34-His170 ion pair (Topham et al., supra). Most of the residues implicated in the proteolytic activity of papain are conserved in Der p 1, including the cysteine and histidine residues of the active site. Although the cysteine protease activity of Der p 1 is generally accepted, studies have revealed that it exhibits a unique mixed cysteine/serine protease activity, even though it has only one active site (Hewitt, C. R. A., et al (1997). Clin. Exp. Allergy, 27: 201-207). The preferred cleavage site is glutamate for the cysteine protease activity and arginine for the serine protease activity.

Der p 1 was shown to cleave CD23 (FcεR II), the low affinity IgE receptor (Hewitt. C., et al (1995). J. Exp. Med., 182: 1537-1544; Schulz, O., et al. (1997). Eur. J. Immunol., 27: 584-588) involved in the regulation of IgE synthesis, thus stimulating IgE production. On the other hand it cleaves CD25, the α subunit of the IL-2 receptor (Schulz, O., et al (1998). J. Exp. Med., 187: 271-275). As IL-2 is a cytokine involved in the propagation of a Th1 immune response, the digestion of its receptor results in skewing towards a Th2 response. Proteolytic activity of Der p 1 has also been shown to enhance Th2 cytokine release from human T cells (Ghaemmaghami, A. M., et al. (2001). Eur. J. Immunol., 31: 1211-1216), and allow an adjuvant activity for a bystander allergen (Ghough L., et al. (2001). Clin. Exp Allergy, 31: 1594-1598).

Moreover, Der p 1 was able to cleave DC-SIGN, DC-SIGNR (Furmonaviciene R, et al, Clin Exp Allergy. 2007 February; 37(2): 231-42) CD40 (Ghaemmaghami A M, et al, Clin Exp Allergy. 2002 October; 32(10): 1468-75), tight junctions proteins (Wan H et al, J Clin Invest. 1999 July; 104(1): 123-33) and alpha-antitrypsin (Kalsheker N A, et al, Biochem Biophys Res Commun. 1996 Apr. 5; 221(1): 59-61.) these cleavages can promote/amplify the TH2-biased allergic response. Furmonaviciene R, Ghaemmaghami A M, Boyd S E, Jones N S, Bailey K, Willis A C, Sewell H F, Mitchell D A, Shakib F. The protease allergen Der p 1 cleaves cell surface DC-SIGN and DC-SIGNR: experimental analysis of in silico substrate identification and implications in allergic responses. Clin Exp Allergy. 2007 February; 37(2): 231-42. Ghaemmaghami A M, Gough L, Sewell H F, Shakib F. The proteolytic activity of the major dust mite allergen Der p 1 conditions dendritic cells to produce less interleukin-12: allergen-induced Th2 bias determined at the dendritic cell level. Clin Exp Allergy. 2002 October; 32(10): 1468-75. Wan H, Winton H L, Soeller C, Tovey E R, Gruenert D C, Thompson P J, Stewart G A, Taylor G W, Garrod D R, Cannell M B, Robinson C. Der p 1 facilitates transepithelial allergen delivery by disruption of tight junctions. J Clin Invest. 1999 July; 104(1): 123-33. Kalsheker N A, Deam S, Chambers L, Sreedharan S, Brocklehurst K, Lomas D A. The house dust mite allergen Der p1 catalytically inactivates alpha 1-antitrypsin by specific reactive centre loop cleavage: a mechanism that promotes airway inflammation and asthma. Biochem Biophys Res Commun. 1996 Apr. 5; 221(1): 59-61.

Recombinant Expression of Group I Allergens

Group 1 allergens include both Der p 1 and Der f 1, the major allergen from the house dust mite Dermatophagoides farinae. Der f 1 has a sequence highly similar to Der p 1 and also belongs to the cysteine protease family. Recombinant Der f 1 has been successfully produced in Escherichia coli (Takahashi K. et al., (2000) Int. Arch. Allergy Immunol., 122: 108-114). The product produced in E. coli had the same amino acid sequence as native Der f 1, and further displayed enzymatic and antigenic properties identical to native Der f 1.

In contrast, expression of mature Der p 1 in E. coli as a fusion protein with α-galactosidase, using the pin-point expression vector (Promega), resulted in a catalytically inactive protein (Scobie G. et al., (1994) Biochem. Soc. Trans. 22: 448S). The product showed some immunoreactivity with a panel of monocolonal and polyclonal antibodies but showed no evidence of catalytic activity. In an attempt to obtain active enzyme, a protocol for solubilisation, denaturation and renaturation was adopted that had been successfully used for papain (Taylor M. A. J. et al., (1992) Prot. Eng. 5: 455-459) but this was unsuccessful with Der p 1. The authors therefore conclude that it is important to incorporate the prosequence for proDer folding of the protein in E. coli, and to obtain enzymatically active Der p 1.

WO2004/076481 (GlaxoSmithKine Biologicals s.a.) discloses that substantial amelioration of protein expression was achieved in E. coli when Der p1/ProDer p1/PreProDer p 1, whether mutated or not, was expressed as a maltose binding protein (MBP) fusion protein. The wild-type MBP-ProDer p 1 fusion protein showed significant IgE binding activity in comparison to recombinant ProDer p 1 derivatives with cysteine residue mutations.

### SUMMARY OF THE INVENTION

The present invention relates to novel methods for producing recombinant allergens, including but not limited to proDer p 1, the precursor form of the major protein allergen from Dermatophagoides pteronyssinus Der p 1, and to recombinant allergens produced by such methods. The invention further relates to the use of said recombinant allergens in formulating immunogenic compositions and vaccines effective in the prevention and/or the reduction of allergic responses to specific allergens.

This invention is not to be limited in scope by the specific embodiments described herein. Indeed, various modifications of the invention in addition to those described herein will become apparent to those skilled in the art from the foregoing description. Such modifications are intended to fall within the scope of the appended claims.

Although one embodiment is for methods of producing recombinant allergens, immunogenic compositions, and methods of treating patients with the immunogenic compositions, the present invention can also be used to produce recombinant allergens including, but not limited to, specific grass pollens, tree pollens, dust mite proteins, animal dander saliva, urine, and molds.

In one embodiment, the invention relates to a method for making a recombinant allergen with reduced IgE binding as compared to native allergen comprising the steps of (i) expressing said recombinant allergen in a host cell, and (ii) selecting said recombinant allergen having an increased IgE reducing structure as compared to said recombinant allergen expressed in a wild-type P. pastoris cell or a wild-type CHO cell. In another embodiment, the host cell is selected from the group consisting of: a prokaryotic host cell, an E. coli cell, a modified CHO cell, a modified P. pastoris cell, and any other cells that induce reduction of IgE reactivity or are modified or engineered to induce reduction of IgE reactivity in the proteins they express.

In yet another embodiment of the invention, the recombinant allergen is an allergen with proteolytic activity, such as, but not limited to ProDer p 1, and Der p 1.

In another embodiment of the invention, the IgE reducing structure is selected from the group consisting of: an altered higher order structure, increased β-sheet content, aggregation and, a complete absence of folding that result in an incorrectly folded allergen after purification.

In another embodiment the invention relates to a recombinant allergen with reduced IgE binding as compared to native allergen, which is obtainable by making a recombinant allergen with reduced IgE binding as compared to native allergen comprising the steps of (i) expressing said recombinant allergen in a host cell, and (ii) selecting said recombinant allergen having an increased IgE reducing structure as compared to said recombinant allergen expressed in a wild-type P. pastoris cell or a wild-type CHO cell. In another embodiment, the host cell is selected from the group consisting of: a prokaryotic host cell, an E. coli, a modified CHO cell, and a modified P. pastoris cell.

In another embodiment the invention relates to an immunogenic composition comprising a recombinant allergen with reduced IgE binding as compared to native allergen.

In another embodiment, the invention relates to an immunogenic composition comprising a recombinant allergen further comprising an increased IgE reducing structure as compared to said recombinant allergen expressed in a wild-type P. pastoris or wild-type CHO cell. In yet another embodiment, immunogenic composition further comprises an adjuvant. In another embodiment of the invention, the adjuvant is a preferential stimulator of Th1-type immune responses, such as at least one selected from the group of: 3D-MPL, QS21, a CpG oligonucleotide, a polyethylene ether and a polyethylene ester. Alternatively, the immunogenic composition according to one embodiment of the invention the recombinant allergen is presented in an oil in water or a water in oil emulsion vehicle.

In another embodiment, the invention relates to a method of treating a patient suffering from or susceptible to allergic responses, comprising administering to said individual a recombinant allergen according the invention.

In another embodiment, the invention relates to a method of treating a patient suffering from or susceptible to allergic responses, comprising administering to said individual an immunogenic composition according the invention.

In one embodiment, the invention relates to a method for producing a recombinant Dermatophagoides pteronyssinus ProDer p1 derivative that has significantly reduced allergenic activity compared to that of the native allergen, the method comprising expression of a sequence encoding ProDer p1 in a prokaryotic host cell.

In other embodiments, the invention provides for a method which comprises: culturing a transformed prokaryotic host cell, wherein the cell has been transformed with an expression vector capable of expressing a DNA sequence encoding recombinant ProDer p1 protein in a prokaryotic cell, under conditions permitting expression of said protein; and recovering said protein.

In other embodiments, the invention provides for a method which comprises: culturing a transformed prokaryotic host cell, wherein the cell has been transformed with an expression vector capable of expressing a DNA sequence encoding recombinant ProDer p1 protein in a prokaryotic cell, under conditions permitting expression of said protein; and recovering said protein and purification of the ProDer p 1 protein.

In other embodiments, the invention provides for a method which comprises: culturing a transformed prokaryotic host cell, wherein the cell has been transformed with an expression vector capable of expressing a DNA sequence encoding recombinant ProDer p1 protein in a prokaryotic cell, under conditions permitting expression of said protein; and recovering said protein, purification of the ProDer p 1 protein, denaturing and renaturing the ProDer p 1 protein and/or thermal treatment of the ProDer p 1 protein.

In one embodiment, the invention relates to a method described herein where the prokaryotic host cell is E. coli.

In another embodiment, the invention relates to a method wherein the expression vector is a toxin/antidote plasmid.

In another embodiment, the invention relates to a method wherein the ProDer p 1 sequence corresponds to the wild-type sequence (SEQ ID NO: 3).

In yet another embodiment, the invention relates to a method wherein the ProDer p1 sequence comprises one or more of the following mutations (Der p 1 numbering):

a mutation of the cysteine 31 residue, optionally to arginine or lysine;

a mutation of the cysteine 34 residue, optionally to alanine;

a mutation of the cysteine 65 residue, optionally to arginine or lysine;

a mutation of the cysteine 71 residue, optionally to arginine or lysine;

a mutation of the cysteine 103 residue, optionally to arginine or lysine;

a mutation of the cysteine 117 residue, optionally to arginine or lysine;

a mutation of the histidine 170 residue; and

a mutation at the site of cleavage between the propeptide and the mature molecule, optionally a deletion of the residues NAET.

In another embodiment, the invention relates to the methods described above wherein the ProDer p 1 sequence comprises the three following mutations (Der p 1 numbering): a mutation of the cysteine 71 residue, a mutation of the cysteine 103 residue and a mutation of the cysteine 117 residue.

In another embodiment, the invention relates to the methods described above wherein the ProDer p1 sequence further comprises a deletion of amino acid residues 147 to 160 (Der p 1 numbering) or residues 227-240 (ProDer p1 numbering).

In another embodiment, the invention relates to a method according to any one of claims 1 to 7, wherein the ProDer p1 sequence comprises from 1 to 10 additional amino acids at the C-terminus and/or N-terminus of the wild-type sequence.

In another embodiment, the invention relates to the methods described above wherein the ProDer p 1 protein has the sequence of SEQ ID NO: 1.

In another embodiment, the invention relates to the methods described above wherein the ProDer p 1 is not expressed as a fusion protein.

In another embodiment, the invention relates to the methods described above wherein the sequence encoding ProDer p1 has a codon usage pattern which is optimized for eukaryotic expression.

In another embodiment, the invention relates to a recombinant Dermatophagoides pteronyssinus ProDer p1 derivative that has significantly reduced allergenic activity compared to that of the native allergen, which is obtainable by a process according to any one of the methods described herein.

In another embodiment, the invention relates to an immunogenic composition comprising a recombinant ProDer p1 protein and, optionally, an adjuvant.

In another embodiment, the invention relates to an immunogenic composition wherein the adjuvant is a preferential stimulator of Th1-type immune responses.

In another embodiment, the invention relates to an immunogenic composition wherein the adjuvant comprises one or more of 3D-MPL, QS21, a CpG oligonucleotide, a polyethylene ether or ester or a combination of two or more of these adjuvants.

In another embodiment, the invention relates to an immunogenic composition wherein the allergen is presented in an oil in water or a water in oil emulsion vehicle.

In another embodiment, the invention relates to a method of treating a patient suffering from or preventing a patient susceptible to allergic responses, comprising administering to said individual a recombinant ProDer p 1 protein or an immunogenic composition comprising a recombinant ProDer p1 protein.

In yet another embodiment, the invention relates to a recombinant ProDer p1 derivative having the sequence of SEQ ID NO: 1, an isolated nucleic acid molecule encoding the sequence of SEQ ID NO: 1, and an isolated nucleic acid molecule that comprises, consists essentially of, or consists of the sequence of SEQ ID NO: 2.

In yet another embodiment, the invention relates to an expression vector containing a ProDer p1 nucleic acid sequence.

In yet another embodiment, the invention relates to a host cell transformed with a ProDer p1 nucleic acid sequence or with an expression vector containing a ProDer p1 nucleic acid sequence.

It is to be understood that both the foregoing summary description and the following detailed description are exemplary and explanatory, and are intended to provide further explanation of the invention as claimed.

The accompanying drawings are included to provide a further understanding of the invention, and are incorporated in, and constitute a part of this specification, illustrate several embodiments of the invention, and together with the description serve to explain the principles of the invention.

### BRIEF DESCRIPTION OF THE FIGURES

FIG. 1A graphically illustrates IgE reactivity of the recombinant E. coli ProDer p 1 performed with 24 sera.

FIG. 1B graphically illustrates IgE inhibition assays. Plates were coated with natural Der p 1 (500 ng/well). A pool of 20 human sera from patients with mite allergy (RAST value>100 kU/L) were preincubated overnight at 48° C. with various concentrations (0-200 μg/ml) of Der p 1 or ProDer p 1 produced in CHO or in E. coli as inhibitors. The sera were then added on ELISA plates, and IgE-binding activity was measured as described above.

FIG. 1C graphically illustrates a rat basophil leukemia cell mediator release assay. Rat basophil leukemia cells RBL-SX-38 (received from Prof. Kinet, Beth Israel Deaconess Medical Center, Boston, USA), expressing human FcεRI, were sensitized with serial dilutions of 4 sera from allergic patient to D. pteronyssinus (RAST value>100 kU/L, from 1/3 to 1/96 in base 2 dilution) for 17 h. Triggering of RBL cells was induced by adding 10 ng of purified natural Der p 1 or ProDer p 1 produced in E. coli in degranulation medium (RPMI 1640 without phenol red containing 1 mg/ml BSA) for 30 min at 37° C. As control, cells were also incubated in the absence of allergen to measure spontaneous release. Total release was obtained by adding 0.5% Triton X 100 to the medium. To determine the □-hexosaminidase release activity, 50 μl of the supernatant and 50 μl of p nitrophenyl N acetyl □ D glucosaminide (2 mM in 0.2 M citric acid buffer, pH 4.5) were mixed in a separate 96-well plate for 3 h at 37° C. The reaction was terminated by adding 150 μl of 1M Tris-HCl, pH 9, and the absorbance at 405 nm was measured. Results were expressed as percentage of the total release minus the spontaneous release.

FIG. 2 graphically illustrates results of a Der p1 ELISA, confirming the absence of conformational epitopes in the allergen variant produced in the bacteria.

FIG. 3 graphically illustrates how ProDer p 1 isolated from inclusion bodies maintains Der p 1-specific T cell reactivity.

FIG. 4 graphically illustrates how the prophylactic potential of ProDer p 1 coli was compared with that of ProDer p 1 produced in CHO cells in a Der p 1 sensitization model developed in Balb/c mice. On days 0,14 and 28, groups of 7 mice were intramuscularly (with MPL) or intraperitoneally (with alum) vaccinated with purified ProDer p 1 coli or CHO. Control group (group 2) was not vaccinated. Three weeks after the last vaccination, all the mice were sensitized with Der p 1 adjuvanted with alum. To induce airway inflammation, mice were challenged 13 days after the last sensitization by exposures to aerosolized crude D. pteronyssinus extracts containing 10 μg/ml Der p 1.

FIG. 5 graphically illustrates ProDer p 1 coli vaccinations induced weak specific IgE responses when adjuvanted with MPL. No specific IgE is observed with ProDer p 1 coli adjuvanted with alum. ProDer p 1 CHO induced the production of specific IgE response. After sensitization with Der p 1/alum, the specific IgE is increasing in mice vaccinated with ProDer p 1 coli but the response is much weaker than that measured in control animals (mice not vaccinated but sensitized with Der p 1/alum). The IgE response obtained with ProDer p 1 CHO/alum is comparable with that of control animals. When ProDer p 1 CHO is adjuvanted with MPL, we obtained a specific IgE response comparable with those obtained with ProDer p 1 coli. It is likely due to the effect of MPL (a Th1 adjuvant: an anti-IgE adjuvant). Consequently, the comparison of the IgE response between ProDerp1 coli and ProDer p 1 CHO adjuvanted with alum (a Th2 adjuvant, a Pro-IgE adjuvant) clearly demonstrates the weaker IgE reactivity of ProDer p 1 coli.

FIG. 6 graphically illustrates ProDer p 1 coli vaccinations induced lower specific IgG1 response compared with ProDer p 1 CHO. The production of specific IgG1 is a marker of Th2 response is markedly dependent on the type of adjuvant used. Alum (Pro-Th2) must induce more IgG1 than MPL (pro-Th1 adjuvant). But, after sensitizations, the specific IgG1 response is weaker in mice vaccinated with ProDer p 1 coli. The difference of specific IgG1 response between ProDer p 1 coli-vaccinated animals and animals only sensitized with Der p 1/alum results from the number of allergen injections. ProDer p 1 coli vaccinated animals received 3 injections of ProDer p 1 coli+2 injections of Der p 1. Control animals received only 3 injections of Der p 1. This figure indicates that ProDer p 1 coli is less immunogenic than ProDer p 1 CHO.

FIG. 7 graphically illustrates that the production of specific IgG2a is a marker of Th1 response is markedly dependent on the type of adjuvant used. Alum (Pro-Th2) must induce less IgG2a than MPL (pro-Th1 adjuvant). Specific IgG2a is only observed after vaccinations with MPL (for ProDer p 1 coli and ProDer p 1 CHO). Again, the specific IgG1 response is weaker in mice vaccinated with ProDer p 1 coli. This production of IgG2a is maintained even after the 3 sensitization injections with Der p 1/alum. This figure confirmed that ProDer p 1 coli is less immunogenic than ProDer p 1 CHO. FIG. 8 graphically illustrates that ProDer p 1 coli retained the T-cell reactivity of Der p 1.

FIG. 9 graphically illustrates that the production of IFNg is a marker of Th1 whereas the secretion of IL-5 is a marker of Th2 response. These cytokine productions must be dependent on the type of adjuvant used. Alum (Pro-Th2) must induce more IL-5 and less IFNg than MPL (pro-Th1 adjuvant). As expected, control mice secreted only IL-5 (allergic). Vaccination with ProDer p 1 coli induced the production of IL-5 and INFg, as ProDer p 1 CHO. However, the production of IL-5 is much higher in group vaccinated with ProDer p 1 CHO/alum. The IL-5 production induced by ProDer p 1 coli/MPL is weaker than that of the control group. This figure indicates that ProDer p 1 coli can induce pro-Th1 and pro-Th2 cytokines.

FIG. 10 graphically illustrates that there is a reduction of eosinophilia in vaccinated groups for both recombinant allergens and for both adjuvants.

FIG. 11 graphically illustrates if the total number of eosinophils (eosinophil percentage×total number of cells in BAL) are counted, it is clear that ProDer p 1 coli or CHO vaccinations reduced drastically the eosinophil infiltrates (inflammation reduction).

FIG. 12 graphically illustrates that after the challenge with aerosolized house dust mite extracts and just before the sacrifice, the airway hyperresponsiveness (AHR) was measured with methacholine stimulation. High values of AHR were measured in control animals (G2 Der p 1 endo low). Vaccination with ProDer p 1 coli/alum or ProDer p 1 CHO/alum reduced the AHR. This reduction is not observed when MPL was used as adjuvant. This figure indicates ProDer p 1 coli can reduce the AHR.

### DETAILED DESCRIPTION OF THE INVENTION

The present inventors have now found that the IgE reactivity of recombinant ProDer p 1 expressed in a prokaryotic cell is drastically reduced compared to Der p 1 and ProDer p 1 expressed in mammalian cells. A Der p 1 ELISA based on specific monoclonal antibodies confirmed the absence of conformational epitopes in the allergen variant produced by the bacteria. However, the isolated ProDer p 1 maintained Der p 1-specific T cell reactivity. Furthermore, it was possible to obtain high expression levels of ProDer p 1 in E. coli without the use of a fusion protein.

Accordingly, one embodiment of the present invention provides a method for producing a recombinant ProDer p 1 derivative that has significantly reduced allergenic activity compared to that of the native allergen, the method comprising expression of the ProDer p 1 sequence in a prokaryotic cell. The term “native” is used herein to mean the allergen in the form produced by house dust mites.

A further embodiment of the invention relates to a recombinant allergen, such as but not limited to ProDer p 1, an enzymatically inactive Der p 1 precursor form, produced in E. coli displayed very low IgE binding capacity but retained its T-cell reactivity. To validate this hypoallergenic character, the application discloses the prophylactic potential of ProDer p 1 produced in a host cell, such as but not limited to, E. coli in a Der p 1 sensitization murine model.

In another embodiment of the invention, Gel filtration chromatography as well as FTIR spectroscopy demonstrated that ProDer p 1 is produced in E. coli as aggregates containing an higher β-sheet content than ProDer p 1 produced in wild-type P. pastoris or in wild-type CHO cells. These conformational changes explain the drastic reduction of its in vitro IgE binding activity towards human allergic sera. Compared with ProDer p 1 produced in wild-type CHO, vaccination of native mice with ProDer p 1 coli adjuvanted with alum induced a mixed Th1-Th2 immune response characterized by the weak production of specific IgG2a, IgG1 antibodies and the absence of the specific IgE titers.

Unpredictably, the drastic reduction of specific IgE titers was maintained after mice sensitizations with natural Der p 1/alum and subsequent challenges with aerosolized house dust mite extracts. Moreover, the Th1-Th2 bias was confirmed as vaccination with ProDer p 1 coli induced the secretion of IFNγ and IL-5 whereas the control allergic group secreted only IL-5. ProDer p 1 coli prevented the development of airway eosinophilia following house dust mite extract challenges of immunized mice. Furthermore, the increase in airways sensitivity to inhaled methacholine was reduced by the prophylactic vaccination. This embodiment of the present invention indicates that ProDer p 1 coli could represent an hypoallergen suitable for the prevention or treatment of house dust mite allergy.

One embodiment of the invention, is a method for making a recombinant allergen with reduced IgE binding as compared to native allergen comprising the steps of (i) expressing said recombinant allergen in a host cell, and (ii) selecting said recombinant allergen having an increased IgE reducing structure as compared to said recombinant allergen expressed in a wild-type P. pastoris cell or a wild-type CHO cell. In another embodiment of the invention, the recombinant allergen is an allergen with proteolytic activity. Examples of allergens with proteolytic activities include, but are not limited to, Mold allergens, such as Penicillium citrinum, pen C 13, an allergenic glycoprotein Epicoccum purpurascens Epi p 1, Dermatophagoides pteronyssinus (dust mite), Der p 1, Der p 3, Der p 6, Der p 9, Dermatophagoides farinae, Der f 1, Der f 3, Der f 6, Der f 9, Blomia tropicalis, Blo t 1, Blo t 3, Rhodotorula mucilaginosa (yeast), Rho m 2, cockroach Bla g 2, Curvularia lunata (fungi), Cur 11, Penicillium chrysogenum (fungi), Pen ch 18, Phleum pretense (grass pollen), Phl p 1.

Another method of the invention may be performed by conventional recombinant techniques such as described in Maniatis et. al., Molecular Cloning-A Laboratory Manual; Cold Spring Harbor, 1982-1989. In particular, the process may comprise the steps of:
(a) culturing a transformed prokaryotic host cell, wherein the cell has been transformed with an expression vector capable of expressing a DNA sequence encoding recombinant ProDer p 1 protein in a prokaryotic cell, under conditions permitting expression of said protein; and
(b) recovering said protein.

The term ‘transforming’ is used herein to mean the introduction of foreign DNA into a host cell by transformation, transfection or infection with an appropriate plasmid or viral vector using e.g. conventional techniques as described in Genetic Engineering; Eds. S. M. Kingsman and A. J. Kingsman; Blackwell Scientific Publications; Oxford, England, 1988. The term ‘transformed’ or ‘transformant’ will herein apply to the resulting host cell containing and expressing the foreign gene of interest.

The expression vector may be an integrating or replicable expression vector. The expression vector may be prepared by cleaving a vector compatible with a prokaryotic host cell to provide a linear DNA segment having an intact replicon, and combining said linear segment with one or more DNA molecules which, together with said linear segment encode the desired product, such as the DNA polymer encoding the ProDer p 1 protein, under ligating conditions. Thus, the DNA polymer may be preformed or formed during the construction of the vector, as desired.

In one embodiment of the invention, any suitable prokaryotic (bacterial) host cell may be used for expression of the protein. In another embodiment the prokaryotic host cell used in the method is E. coli. In yet another embodiment of the invention, ProDer p 1 expressed in E. coli resulted in an incorrectly folded allergen after purification. The protein displays a folding characterized by a higher β-sheet content (characteristics of protein aggregates). In another embodiment of the invention, when ProDer p 1 is expressed in wild-type CHO cells, ProDer p 1 adopts a correct folding. Therefore, it should be appreciated that one skilled in the art could modify a CHO, P. pastoris, or other cell, to express an incorrectly folded recombinant allergen having similar characteristics as seen with proDer p 1 expressed in E. coli, such as reduced IgE binding as compared to native allergen. Other representative examples of appropriate hosts cells include bacterial cells, such as cells of streptococci, staphylococci, enterococci, E. coli, streptomyces, cyanobacteria, Bacillus subtilis, and Streptococcus pneumoniae; fungal cells, such as cells of a yeast, Kluveromyces, Saccharomyces, a basidiomycete, Candida albicans and Aspergillus; insect cells such as cells of Drosophila S2 and Spodoptera Sf9; animal cells such as modified (non wild-type) CHO, modified (non wild-type) P. pastoris, COS, HeLa, C127, 3T3, BHK, 293, CV-1 and Bowes melanoma cells; and plant cells, such as cells of a gymnosperm or angiosperm.

Any vector suitable for use in the selected prokaryotic host cell may be used in the method of the invention. Suitable vectors include plasmids, bacteriophages, cosmids and recombinant viruses. According to one embodiment, a plasmid toxin-antidote system may be used, for example the pStaby1 expression vector (Delphi Genetics). The plasmid toxin-antidote system typically uses an antidote gene in the plasmid DNA, normally under the control of a constitutive promoter. The gene encoding the toxic product (or poison) is typically introduced into the chromosome of the bacteria into which the plasmid will be introduced. Expression of the poison gene is generally under the control of a promoter that is repressed by the antidote protein, so that when the plasmid is present in the bacteria, the toxin may not be expressed. If however the plasmid is lost from the bacteria, the antidote may be degraded and the production of the toxin may be induced, causing cell death. This system aims to eliminate all plasmid-free cells from the population, irrespective of the manner by which the plasmid was lost, thus ensuring plasmid maintenance.

The preparation of the replicable expression vector may be carried out conventionally with appropriate enzymes for restriction, polymerisation and ligation of the DNA, by procedures described in, for example, Maniatis et al cited above. The recombinant host cell may be prepared by transforming a prokaryotic host cell with a replicable expression vector encoding ProDer p 1 under transforming conditions. Suitable transforming conditions are conventional and are described in, for example, Maniatis et al cited above, or “DNA Cloning” Vol. II, D. M. Glover ed., IRL Press Ltd, 1985.

The choice of transforming conditions is determined by the host cell. Thus, a bacterial host such as E. coli may be treated with a solution of CaCl₂ (Cohen et al, Proc. Nat. Acad. Sci., 1973, 69, 2110) or with a solution comprising a mixture of RbCl, MnCl₂, potassium acetate and glycerol, and then with 3-[N-morpholino]-propane-sulphonic acid, RbCl and glycerol.

Culturing the transformed host cell under conditions permitting expression of the DNA polymer is carried out conventionally, as described in, for example, Maniatis et al and “DNA Cloning” cited above. Thus, typically the cell is supplied with nutrient and cultured at a temperature below 45° C.

The product may be recovered by conventional methods, for example the host cell may be lysed physically, chemically or enzymatically and the protein product isolated from the resulting lysate. Conventional protein isolation and purification techniques include selective precipitation, absorption chromatography, and affinity chromatography, for example using a monoclonal antibody affinity column.

According to one embodiment of the invention, the ProDer p 1 may be produced as an insoluble protein within an inclusion body. The inclusion bodies are typically isolated for the extraction of ProDer p 1, for example using centrifugation. The extraction process may comprise a step of solubilising the protein. The extraction of protein from inclusion bodies may be carried out in the presence of urea, for example in the range of 4 to 8 M urea, such as about 6 M urea. Optionally the extraction medium may further comprise sodium chloride, for example from 200 to 400 mM NaCl, such as about 300 mM NaCl. Typically extraction is carried out at a pH of from 7 to 8, for example approximately pH 7.5. The extracted protein may be purified using a Ni2+ column, optionally under denaturing conditions. The ProDer p1 protein may then be renatured, for example by using dialysis to remove urea.

The ProDer p 1 protein produced by the method of the invention may further be thermally treated. For example, the protein may be thermally treated for a few minutes, e.g. about 5 minutes, at a temperature of about 100° C. The protein is optionally thermally treated in the presence of a reducing agent such as beta-mercaptoethanol or DTT. This type of treatment typically has a detrimental effect on the stability of protein conformational IgE-binding epitopes, and thus may further reduce the IgE-reactivity of the resulting protein. The thermal treatment may thus be used to denature the protein.

The ProDer p 1 sequence expressed according to the present invention may correspond to the wild type sequence or may be a mutated sequence as described herein. According to one embodiment, the ProDer p 1 protein is not expressed as a fusion protein (such as a fusion of ProDer p 1 with maltose binding protein (MBP) or with β-galactosidase). In other words, the protein expressed by the prokaryotic cell consists solely of the ProDer p1 protein sequence. However, the ProDer p 1 protein sequence may include a number of additional amino acids at either the N- or C-terminus compared to the wild-type sequence, as discussed herein, without being considered to be a fusion protein.

Mutations may be introduced into the wild-type sequence before recombinantly producing the hypoallergenic mutants. For example, the sequence may be mutated in order to further reduce the allergenic activity of the ProDer p 1 derivative produced. Such mutations may comprise substitutions, deletions, or additions to the wild type sequence or alterations in the three dimensional structure of the protein such that the tridimensional conformation of the protein is lost. This may be achieved, for example, by deleting cysteine residues involved in disulphide bridge formation or by deleting or adding residues such that the tertiary structure of the protein is substantially altered.

Mutations may be generated with the effect of altering the interaction between two cysteine residues, typically one mutation at positions 4, 31, 34, 65, 71, 103 and 117 of the mature Der p 1 (which corresponds to positions 84, 111, 114, 145, 151, 183 and 197 of ProDer p 1, respectively). Such a mutated protein may comprise two or more (3, 4, 5 or all 6) cysteine mutations, thereby affecting different disulphide bridges, such as mutations at positions 4 & 31, 4 & 65, 4 & 71, 4 & 103, 31 & 65, or 4 & 31 & 65, or at positions 71 & 103, 71 & 117, 103 & 117, 31 & 117, 65 & 117, or 71 & 103 & 117. The derivatives may comprise one single mutation at any of the above positions. In one embodiment, the mutation involves Cys4 (or alternatively, or in addition, Cys117 which is thought to be the disulphide bond partner of Cys4). The Cys mutations may be deletions or substitutions for any of the other natural 19 amino acids. For example, substitutions may introduce positively charged amino acid residues to further destabilise the 3D-structure of the resulting protein, such as cysteine to arginine or lysine substitution. The mutation may be a mutation of the histidine 170 residue.

In one embodiment of the present invention, the derivatives comprise a triple mutation in which the cysteine residues 71, 103 and 117 are all mutated, optionally to alanine. In a further aspect, the amino acids 227-240 of the ProDer p 1 sequence are deleted. These amino acids correspond to 147-160 of the Der p 1 sequence. In a yet further aspect, a cysteine residue is substituted for an arginine residue at position Cys4 of the Der p 1 protein sequence. In another aspect, a cysteine residue is substituted for an arginine residue at any of the following positions (calculated by reference to the sequence in mature Der p 1): Cys31 of Der p 1 protein sequence, Cys65, Cys71, Cys103 or Cys117.

In one embodiment of the invention, the encoding cDNA is mutated so that it encodes additional amino acid residues at the C-terminus, at the N-terminus or at both the C-terminus and N-terminus compared to the wild-type ProDer p 1 amino acid sequence. Accordingly, the recombinant ProDer p 1 amino acid sequence may contain from 1 to 10 additional amino acid residues at the C-terminus and/or N-terminus of the wild-type sequence, for example up to an additional 2, 3, 4, 5, 6, 7, 8, 9 or 10 amino acid residues at one or both termini. Such additional amino acids do not fall within the scope of a fusion protein. These extra amino acids may comprise or be in addition to a series of histidine residues, such as 6 consecutive histidines (histidine tag). Such a series of histidine residues may be included to aid purification of the recombinant protein.

According to another embodiment of the invention, the recombinant amino acid sequence comprises 3 additional amino acid residues at the N-terminus of the native sequence. For example, the recombinant protein sequence may comprise the additional amino acid sequence MAS at the N-terminus of the native sequence, or a variant of this sequence that contains one or more conservative substitutions.

According to another embodiment of the invention, the recombinant amino acid sequence comprises 5 additional amino acid residues at the C-terminus of the native sequence. For example, the recombinant protein sequence may comprise the additional amino acid sequence RRREL at the C-terminus of the native sequence, or a variant of this sequence that contains one or more conservative substitutions. In a yet further aspect, the recombinant protein comprises both an additional 3 amino acid residue sequence at the N-terminus and an additional 5 amino acid residue sequence at the C-terminus, such as those described herein.

Accordingly, the invention provides a ProDer p 1 derivative consisting of the sequence as set out in SEQ ID NO: 1. This sequence comprises additional sequence at both the N- and C-terminus. In another embodiment, the derivative sequence may further comprise a 6-histidine tag.

Mutated versions of ProDer p 1 may be prepared by site-directed mutagenesis of the cDNA which codes for the ProDer p 1 protein by conventional methods such as those described by G. Winter et al in Nature 1982, 299, 756-758 or by Zoller and Smith 1982; Nucl. Acids Res., 10, 6487-6500, or deletion mutagenesis such as described by Chan and Smith in Nucl. Acids Res., 1984, 12, 2407-2419 or by G. Winter et al in Biochem. Soc. Trans., 1984, 12, 224-225.

A further embodiment of the present invention provides an isolated nucleic acid encoding SEQ ID NO: 1 as disclosed herein, for example having the sequence of SEQ ID NO: 2. The nucleotide sequence is typically a DNA sequence and may be synthesized by standard DNA synthesis techniques, such as by enzymatic ligation as described by D. M. Roberts et al in Biochemistry 1985, 24, 5090-5098, by chemical synthesis, by in vitro enzymatic polymerization, or by a combination of these techniques. The nucleic acid sequence may have a codon usage pattern that has been optimised so as to mimic the one used in the intended expression host, namely in a prokaryotic host cell.

Enzymatic polymerisation of DNA may be carried out in vitro using a DNA polymerase such as DNA polymerase I (Klenow fragment) in an appropriate buffer containing the nucleoside triphosphates dATP, dCTP, dGTP and dTTP as required at a temperature of 10°-37° C., generally in a volume of 50 ml or less. Enzymatic ligation of DNA fragments may be carried out using a DNA ligase such as T4 DNA ligase in an appropriate buffer, such as 0.05M Tris (pH 7.4), 0.01M MgCl₂, 0.01M dithiothreitol, 1 mM spermidine, 1 mM ATP and 0.1 mg/ml bovine serum albumin, at a temperature of 4° C. to ambient, generally in a volume of 50 ml or less. The chemical synthesis of the DNA polymer or fragments may be carried out by conventional phosphotriester, phosphite or phosphoramidite chemistry, using solid phase techniques such as those described in ‘Chemical and Enzymatic Synthesis of Gene Fragments-A Laboratory Manual’ (ed. H. G. Gassen and A. Lang), Verlag Chemie, Weinheim (1982), or in other scientific publications, for example M. J. Gait, H. W. D. Matthes, M. Singh, B. S. Sproat, and R. C. Titmas, Nucleic Acids Research, 1982, 10, 6243; B. S. Sproat and W. Bannwarth, Tetrahedron Letters, 1983, 24, 5771; M. D. Matteucci and M. H Caruthers, Tetrahedron Letters, 1980, 21, 719; M. D. Matteucci and M. H. Caruthers, Journal of the American Chemical Society, 1981, 103, 3185; S. P. Adams et al., Journal of the American Chemical Society, 1983, 105, 661; N. D. Sinha, J. Biernat, J. McMannus, and H. Koester, Nucleic Acids Research, 1984, 12, 4539; and H. W. D. Matthes et al., EMBO Journal, 1984, 3, 801.

Alternatively, the coding sequence can be derived from ProDer p 1 mRNA, using known techniques (e.g. reverse transcription of mRNA to generate a complementary cDNA strand), and commercially available cDNA kits.

The DNA code has 4 letters (A, T, C and G) and uses these to spell three letter “codons” which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons.

Where more than one codon is available to code for a given amino acid, it has been observed that the codon usage patterns of organisms are highly non-random. Different species show a different bias in their codon selection and, furthermore, utilization of codons may be markedly different in a single species between genes which are expressed at high and low levels. This bias is different in viruses, plants, bacteria, insect and mammalian cells, and some species show a stronger bias away from a random codon selection than others. For example, humans and other mammals are less strongly biased than certain bacteria or viruses. For these reasons, there is a significant probability that a mammalian gene expressed in E. coli or a viral gene expressed in mammalian cells will have an inappropriate distribution of codons for efficient expression. However, a gene with a codon usage pattern suitable for E. coli expression may also be efficiently expressed in humans. It is believed that the presence in a heterologous DNA sequence of clusters of codons which are rarely observed in the host in which expression is to occur, is predictive of low heterologous expression levels in that host.

There are several examples where changing codons from those which are rare in the host to those which are host-preferred (“codon optimisation”) has enhanced heterologous expression levels, for example the BPV (bovine papilloma virus) late genes L1 and L2 have been codon optimised for mammalian codon usage patterns and this has been shown to give increased expression levels over the wild-type HPV sequences in mammalian (Cos-1) cell culture (Zhou et. al. J. Virol 1999. 73, 4972-4982). In this work, every BPV codon which occurred more than twice as frequently in BPV than in mammals (ratio of usage>2), and most codons with a usage ratio of >1.5 were conservatively replaced by the preferentially used mammalian codon. In WO97/31115, WO97/48370 and WO98/34640 (Merck & Co., Inc.) codon optimisation of HIV genes or segments thereof has been shown to result in increased protein expression and improved immunogenicity when the codon optimised sequences are used as DNA vaccines in the host mammal for which the optimisation was tailored. Therefore, in one embodiment of the invention the nucleic acid sequence encoding ProDer p1 has a codon usage pattern which is optimized for eukaryotic expression, for example mammalian expression, such as for expression in Chinese hamster ovary (CHO) cells.

According to another embodiment, the present invention relates to a recombinant ProDer p 1 derivative that has significantly reduced allergenic activity compared to that of the native allergen, which is obtainable by a method of the invention. The allergenic activity, and consequently the reduction in the allergenic activity, of the recombinant ProDer p 1 derivatives produced by the method of the invention may be compared to the native protein by histamine release activity or by IgE-binding reactivity, for example according to the method detailed in the Example section.

“Substantially reduced allergenic activity” means that the allergenic activity as measured by residual IgE-binding activity is reduced to a maximum of 50% of the activity of the native protein, for example to a maximum of 20%, to a maximum of 10%, to a maximum of 5%, or to less than 5%. Alternatively, “substantially reduced allergenic activity” can also be assessed by measuring the histamine release activity of the mutant. A substantial reduction in activity is when there is a reduction of at least a 100-fold factor as compared to the native protein, for example by a factor of 1000-fold, such as by a factor of 10000-fold.

An “IgE reducing structure” means that the structure of the expressed allergen protein includes but is not limited to, at least one characteristic selected from the group of: an altered higher order structure (for example, an altered 1°, 2°, or 3° structure), increased β-sheet content, aggregation and, a complete absence of folding that result in an incorrectly folded allergen after purification. Such characteristics result in, for example, aggregation and possibly loss of some B cell epitopes, and/or a folding that reduces the availability of proper IgE epitopes.

The immunogenicity of the recombinant derivative may be compared to that of the native allergen by various immunological assays. The cross-reactivity of the derivative and native allergens may be assayed by in vitro T-cell assays after vaccination with either derivative or native allergens. For example, splenic T-cells isolated from vaccinated animals may be restimulated in vitro with either recombinant derivative or native allergen followed by measurement of cytokine production with commercially available ELISA assays, or proliferation of allergen specific T cells may be assayed over time by incorporation of tritiated thymidine. The immunogenicity may be determined by ELISA assay, the details of which may be easily determined by the man skilled in the art.

At least two types of ELISA assay are envisaged. First, to assess the recognition of the ProDer p 1 derivative by sera of mice immunized with the wild type Der p 1; and secondly by recognition of wild type Der p 1 allergen by the sera of HDM allergic patients. Typically each well is coated with approximately 500 ng of purified wild type or mutated Der p 1 overnight at 4° C. After incubating with a blocking solution (for example, TBS-Tween 0.1% with 1% BSA) successive dilutions of sera may be incubated at approximately 37° C. for about 1 hour. The wells are washed, typically at least 5 times, and total IgG may be revealed by incubating with an anti-IgG antibody conjugated with alkaline phosphatase.

The recombinant forms of ProDer p 1 obtainable by a method of the invention may be used as prophylactic or therapeutic vaccines. Said allergen derivatives may have the following advantages over the unaltered wild-type allergen: 1) increases the Th1-type aspect of the immune responses (higher IgG2a for example) in comparison to those stimulated by the wild type allergen, thereby leading to the suppression of allergic potential of the vaccinated host, 2) having reduced allergenicity while still retaining T cell reactivity, thus being more suitable for systemic administration of high doses of the immunogen, 3) will induce Der p 1 specific IgG which compete with IgE for the binding of native Der p 1, 4) efficiently protects against airway eosinophilia even after exposure to aerosolised allergen extract. Such derivatives are suitable for use in therapeutic and prophylactic vaccine formulations which are suitable for use in medicine and more particularly for the treatment or prevention of allergic reactions.

Pharmaceutical, immunogenic and vaccine compositions comprising a hypoallergenic ProDer p 1 derivative produced according to a method of the invention are also provided.

The pharmaceutical compositions of the present invention may include adjuvant compounds, or other substances which may serve to increase the immune response induced by the protein.

The vaccine composition of the invention may comprise an immunoprotective amount of the recombinant version of the ProDer p 1 produced by a method of the invention. The term “immunoprotective” refers to the amount necessary to elicit an immune response against a subsequent challenge such that allergic disease is averted or mitigated. In the vaccine of the invention, an aqueous solution of the protein can be used directly. Alternatively, the protein, with or without prior lyophilization, can be mixed, adsorbed, or covalently linked with any of the various known adjuvants.

Suitable adjuvants are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminium salts such as aluminium hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, and chemokines may also be used as adjuvants.

In the formulations of the invention it is desirable that the adjuvant composition induces an immune response predominantly of the Th1 type. High levels of Th1-type cytokines (e.g., IFN-γ, TNFα, IL-2 and IL-12) tend to favor the induction of cell mediated immune responses to an administered antigen. According to one embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Accordingly, suitable adjuvants that may be used for eliciting a predominantly Th1-type response include, for example, a combination of monophosphoryl lipid A, such as 3-de-O-acylated monophosphoryl lipid A (3D-MPL) together with an aluminium salt. 3D-MPL or other toll like receptor 4 (TLR4) ligands such as aminoalkyl glucosaminide phosphates as disclosed in WO9850399, WO0134617 and WO03065806 may also be used alone to generate a predominantly Th1-type response. Other known adjuvants that may preferentially induce a TH1 type immune response include CpG containing oligonucleotides. The oligonucleotides are characterised in that the CpG dinucleotide is unmethylated. Such oligonucleotides are well known and are described in, for example WO 96/02555. Other suitable adjuvants are other TLR 9 ligands such as CpR containing oligonucleotides as described in EP1322656 and US 2004/0097719. Immunostimulatory DNA sequences are also described, for example, by Sato et al., Science 273: 352, 1996. CpG-containing oligonucleotides may also be used alone or in combination with other adjuvants. For example, an enhanced system involves the combination of a CpG-containing oligonucleotide and a saponin derivative particularly the combination of CpG and QS21 as disclosed in WO 00/09159 and WO 00/62800. The formulation may additionally comprise an oil in water emulsion and/or tocopherol.

Another adjuvant that may be used is a saponin, for example QS21 (Aquila Biopharmaceuticals Inc., Framingham, Mass.), that may be used alone or in combination with other adjuvants. For example, one adjuvant system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other suitable formulations may comprise an oil-in-water emulsion and tocopherol. A particularly potent adjuvant formulation may involve QS21, 3D-MPL and tocopherol in an oil-in-water emulsion, as described in WO 95/17210. In another embodiment, the adjuvants may be formulated in a liposomal composition.

Other suitable adjuvants include Montanide ISA 720 (Seppic, France), SAF (Chiron, Calif., United States), ISCOMS (CSL), MF-59 (Chiron), Detox (Ribi, Hamilton, Mont.), RC-529 (Corixa, Hamilton, Mont.) and other aminoalkyl glucosaminide 4-phosphates (AGPs).

Accordingly there is provided an immunogenic composition comprising a ProDer p 1 hypoallergenic derivative as disclosed herein and an adjuvant, wherein the adjuvant comprises one or more of 3D-MPL, QS21, a CpG oligonucleotide, a polyethylene ether or ester or a combination of two or more of these adjuvants. The ProDer p 1 hypoallergenic derivative within the immunogenic composition may be presented in an oil in water or a water in oil emulsion vehicle.

Vaccine preparation is generally described in Vaccine Design (“The subunit and adjuvant approach” (eds. Powell M. F. & Newman M. J). (1995) Plenum Press New York). Encapsulation within liposomes is described by Fullerton, U.S. Pat. No. 4,235,877. Conjugation of proteins to macromolecules is disclosed, for example, by Likhite, U.S. Pat. No. 4,372,945 and Armor et al., U.S. Pat. No. 4,474,757.

The amount of the protein of the present invention present in each vaccine dose is typically selected as an amount which induces an immunoprotective response without significant, adverse side effects in typical vaccines. Such amount will vary depending upon which specific immunogen is employed and whether or not the vaccine is adjuvanted. Generally, it is expected that each dose will comprise 1-1000 μg of protein, preferably 1-200 μg. An optimal amount for a particular vaccine can be ascertained by standard studies involving observation of antibody titres and other responses in subjects. The vaccines of the present invention may be administered to adults or infants, however, it is preferable to vaccinate individuals soon after birth before the establishment of substantial Th2-type memory responses. Following an initial vaccination, subjects may receive a boost in about 4 weeks, followed by repeated boosts approximately every six months for as long as a risk of allergic responses exists.

Vaccines and pharmaceutical compositions may be presented in unit-dose or multi-dose containers, such as sealed ampoules or vials. Such containers are typically hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

The present invention also provides a process for the production of a vaccine, comprising the steps of producing a recombinant ProDer p 1 derivative according to a method of the invention, purifying the protein and admixing the resulting protein with a suitable adjuvant, diluent or other pharmaceutically acceptable excipient.

The present invention also provides a method for producing a vaccine formulation comprising mixing a protein produced by the method of the present invention together with a pharmaceutically acceptable excipient.

Another embodiment of the invention is the use of a protein as produced by a method of the invention for the manufacture of a vaccine for immunotherapeutically treating a patient susceptible to or suffering from allergy. A method of treating patients susceptible to or suffering from allergy comprising administering to said patients a pharmaceutically active amount of the immunogenic composition disclosed herein is also contemplated by the present invention.

A further embodiment of the invention provides a method of preventing or mitigating an allergic disease in man (particularly house dust mite allergy), which method comprises administering to a subject in need thereof an immunogenically effective amount of a an allergen produced by a method of the invention, or of a vaccine in accordance with the invention.

Vectors, Host Cells, Expression Systems

The invention also relates to vectors that comprise a polynucleotide or polynucleotides of the invention, host cells that are genetically engineered with vectors of the invention and the production of polypeptides of the invention by recombinant techniques. Cell-free translation systems can also be employed to produce such proteins.

Recombinant polypeptides of the present invention may be prepared by processes well known in those skilled in the art from genetically engineered host cells comprising expression systems. Accordingly, in a further aspect, the present invention relates to expression systems that comprise a polynucleotide or polynucleotides of the present invention, to host cells that are genetically engineered with such expression systems, and to the production of polypeptides of the invention by recombinant techniques.

For recombinant production of the polypeptides of the invention, host cells can be genetically engineered to incorporate expression systems or portions thereof or polynucleotides of the invention. Introduction of a polynucleotide into the host cell can be effected by methods described in many standard laboratory manuals, such as Davis, et al., BASIC METHODS IN MOLECULAR BIOLOGY, (1986) and Sambrook, et al., MOLECULAR CLONING: A LABORATORY MANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction and infection.

Representative examples of appropriate hosts cells include bacterial cells, such as cells of streptococci, staphylococci, enterococci, E. coli, streptomyces, cyanobacteria, Bacillus subtilis, and Streptococcus pneumoniae; fungal cells, such as cells of a yeast, Kluveromyces, Saccharomyces, a basidiomycete, Candida albicans and Aspergillus; insect cells such as cells of Drosophila S2 and Spodoptera Sf9; animal cells such as modified (non wild-type) CHO, modified non wild-type P. pastoris, COS, HeLa, C127, 3T3, BHK, 293, CV-1 and Bowes melanoma cells; and plant cells, such as cells of a gymnosperm or angiosperm.

A great variety of expression systems can be used to produce the polypeptides of the invention. Such vectors include, among others, chromosomal-, episomal- and virus-derived vectors, for example, vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses, picornaviruses and retroviruses, and vectors derived from combinations thereof, such as those derived from plasmid and bacteriophage genetic elements, such as cosmids and phagemids. The expression system constructs may comprise control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., MOLECULAR CLONING, A LABORATORY MANUAL, (supra).

Polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography is employed for purification. Well known techniques for refolding protein may be employed to regenerate active conformation when the polypeptide is denatured during isolation and or purification.

The following examples are further illustrative of the present invention. This example is not intended to limit the scope of the present invention, and provides further understanding of the invention.

### EXAMPLES

The invention is further illustrated by way of the following examples which are intended to elucidate the invention. These examples are not intended, nor are they to be construed, as limiting the scope of the invention. Numerous modifications and variations of the present invention are possible in view of the teachings herein and, therefore, are within the scope of the invention. The examples below are carried out using standard techniques, and such standard techniques are well known and routine to those of skill in the art, except where otherwise described in detail.

### Example 1

### Cloning of ProDer p 1 in E. coli Expression Vector

The ProDer p 1 coding cassette from the pNIV4846 plasmid was amplified by PCR using the following primers: 5′GGGGCTAGCCGGCCGAGCTCCATTAAGACC3′ (SEQ ID NO: 4) (Nhe I restriction site in bold and underlined, forward) and 5′GGGGCGGCCGCCAGGATCACCACGTACGGG3′ (SEQ ID NO: 5) (Not I restriction site in bold and underlined, reverse). The amplified fragment was digested with NheI-NotI to generate a 920 bp fragment and introduced into the pStaby1 expression vector (Delphi Genetics) restricted with NheI-NotI. The resulting plasmid contains the ProDer p 1 cassette upstream to a (His)6 sequence tag.

This DNA construct was transformed by electroporation into CYS21 E. coli cells (Dephi Genetics). The presence and identity of the ProDer p 1 cDNA was verified by DNA sequencing. Finally, the recombinant plasmid purified from one CYS21 clone was transformed into SE1 E. coli cells (Delphi Genetics) according to the manual instructions.

Expression and Purification of ProDer p 1 from E. coli

An overnight pre-culture of recombinant SE1 bacteria containing the pStabyl-ProDer p 1 plasmid was used to inoculate 2×500 mL flasks of LB containing 100 μg/ml ampicillin, and cultures were allowed to grow with shaking at 37° C. until an optical density of 0.5 (OD_{600 nm}) was reached. Protein production was induced by adjusting the cell cultures to 1 mM isopropyl-thiogalactoside (IPTG, Duchefa), and the cells were shaken at 37° C. for an additional 2 h. The cells were collected by centrifugation (15 min at 9500 g), resuspended in 40 ml of cold Tris buffer 50 mM pH 7.5, implemented with Aprotinin 1 mM (Sigma) and AEBSF 1 mM (ICN) and lysed by two passages through a cell disrupter (Cell D) at 1800 bars.

The cell lysate was centrifuged for 20 min at 3000 rpm to isolate the inclusion bodies. The pellet was washed with Tris buffer 50 mM pH 7,5, Triton X-100 1% followed by three washing steps to remove the detergent. ProDer p 1 was subsequently extracted overnight at 4° C. with 40 ml of Tris buffer 50 mM, 300 mM NaCl, 6M urea pH 7.5. After ultra centrifugation (45′, 149000 g), the supernatant of extraction was applied at 3 ml/min on a Ni2+ chelateHigh performance column (2.6×6 cm, GE Healthtech Amersham) equilibrated with the extraction buffer. The column was washed with the starting buffer. Protein elution proceeded by step-wise increasing imidazol concentration in the buffer (from 0 to 400 mM). Fractions containing purified ProDer p 1 (elution with 200 mM imidazol) were pooled. ProDer p 1 was renatured by a 2-step dialysis to remove urea (from 6M to 2M urea, from 2M to PBS). The recombinant allergen was concentrated by ultrafiltration (Amicon-Millipore regenerated cellulose ultrafiltration membranes, NMWL 10 kDa) and stored at −20° C.

Der p 1 ELISA

Recombinant ProDer p 1 was detected with an ELISA kit using Der p 1 specific monoclonal antibodies 5H8 and 4C1 (Indoor Biotechnologies, Charlottesville, Va., USA). The Der p 1 standard (UVA 93/03) used in the assay was at a concentration of 2.5 μg/mL.

IgE-Binding Activity

Immunoplates were coated overnight with natural Der p 1 or recombinant ProDer p 1 (500 ng/well) at 4° C. Plates were then washed 5 times with 100 μL per well of TBS-T and saturated for 1 h at 37° C. with 150 μL of the same buffer supplemented with 1% BSA. Sera from allergic patients to D. pteronyssinus and diluted at 1/8 were then incubated for 1 h at 37° C. The specific anti-D. pteronyssinus IgE values (RAST assays) of sera were above the upper cut-off value of 100 kU/L. Plates were washed 5 times with TBS-T buffer and the allergen-IgE complexes were detected after incubation with a mouse anti-human IgE antibody (dilution 1/2000 in TBS-T buffer; Southern Biotechnology Associates, Birmingham, Ala., USA) and a goat anti-mouse IgG antibody coupled to alkaline phosphatase (dilution 1/7500 in TBS-T buffer, Promega). The enzymatic activity was measured using the p-nitrophenylphosphate substrate (Sigma) dissolved in diethanolamine buffer (pH 9.8). OD_{410 nm} was measured in a Biorad Novapath ELISA reader.

T-Cell Reactivity

Spleen cells from Derp 1-immunized mice were stimulated with serial dilutions of ProDer p 1 produced in P. pastoris or in coli. After 72 h, cells were pulsed with 1 μCi/well [³H]thymidine for 16 hours. Cells were harvested and ³H-thymidine uptake was measured by scintillation counting.

Results

Compared with ProDer p 1 produced in CHO (Chinese Hamster ovary cells), ProDer p 1 produced by this expression system carries 3 and 11 extras amino-acid residues at the N- and C-terminus respectively (shown in bold and underlined).

(SEQ ID NO. 6)

MASRP SSIKTFEEYK KAFNKSYATF EDEEAARKNF LESVKYVQSN
GGAINHLSDL SLDEFKNRFL MSAEAFEHLK TQFDLNAETN
ACSINGNAPA EIDLRQMRTV TPIRMQGGCG SCWAFSGVAA
TESAYLAYRN QSLDLAEQEL VDCASQHGCH GDTIPRGIEY
IQHNGVVQES YYRYVAREQS CRRPNAQRFG ISNYCQIYPP
NVNKIREALA QTHSAIAVII GIKDLDAFRH YDGRTIIQRD
NGYQPNYHAV NIVGYSNAQG VDYWIVRNSW DTNWGDNGYG
YFAANIDLMM IEEYPYVVIL RRRELHHHHHH

The nucleic acid sequence of codon-optimised ProDerP1 expressed in E. coli using pStaby1 is shown below (extra sequence shown in bold and underlined).

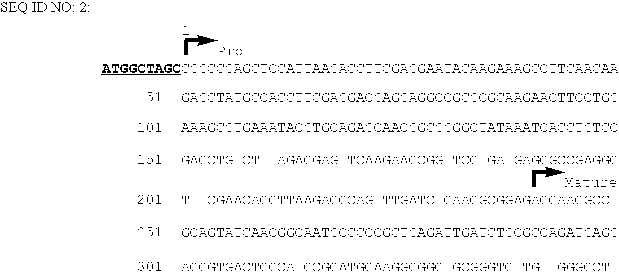

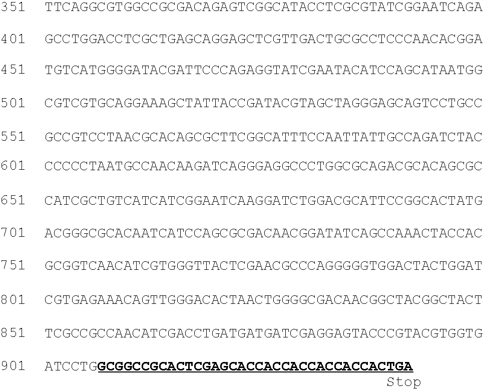

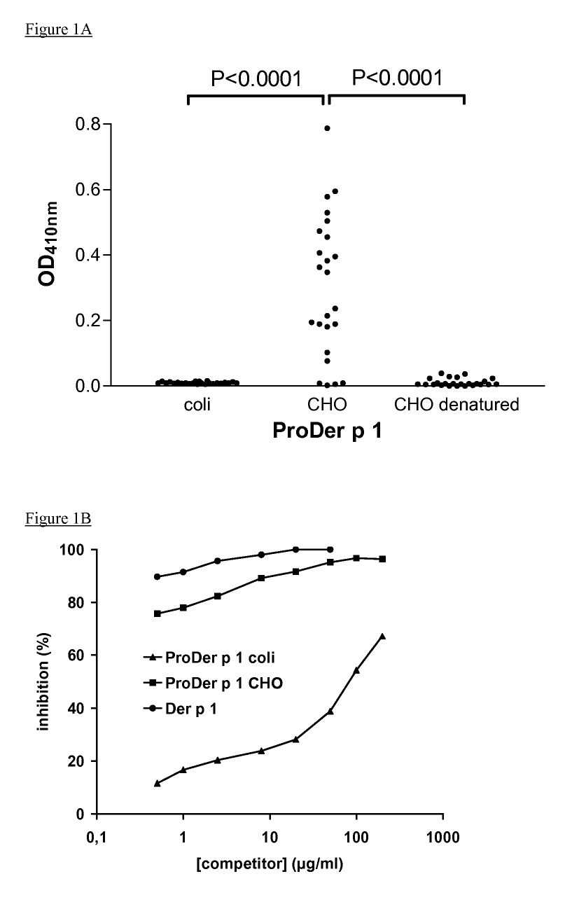

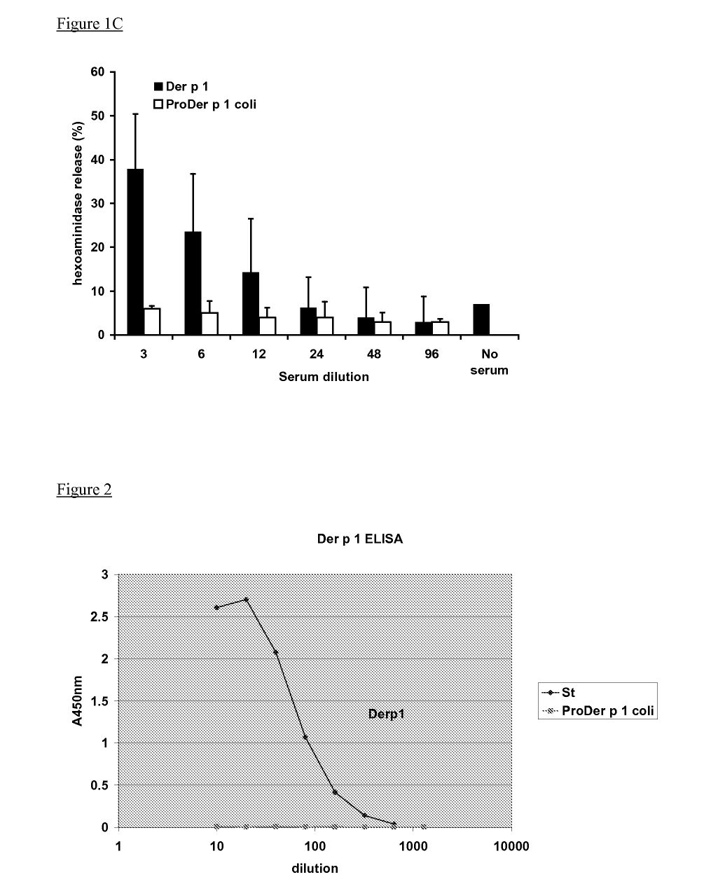

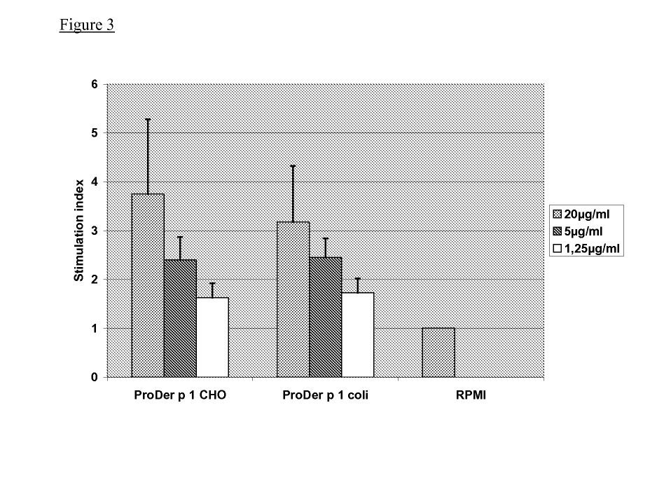

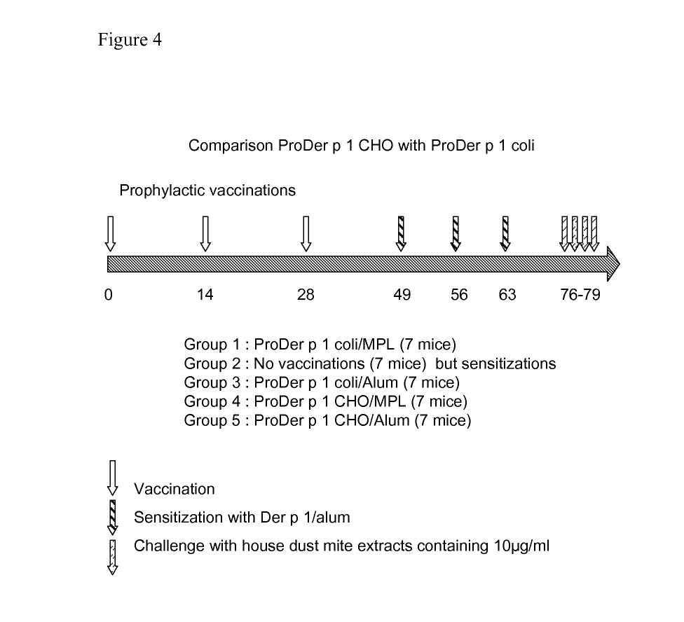

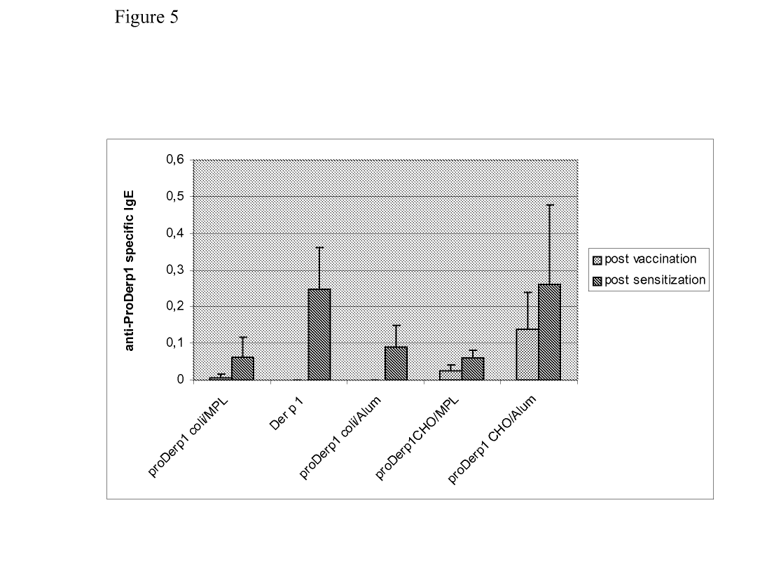

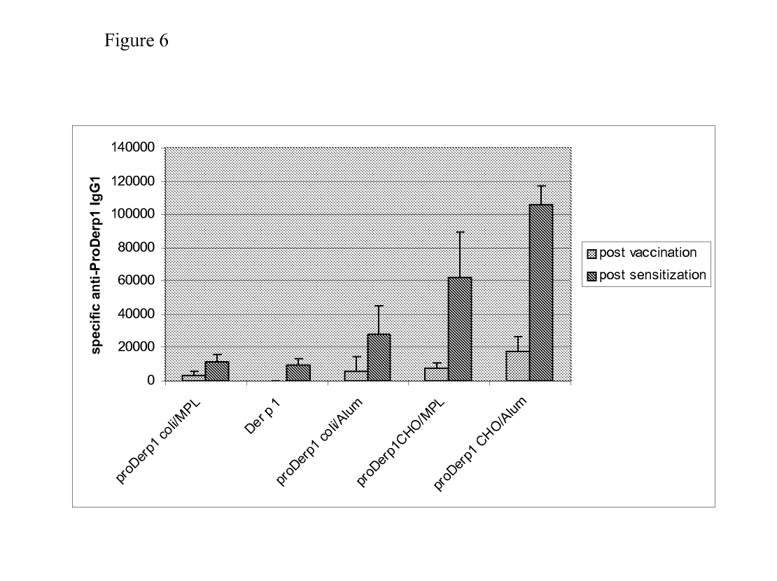

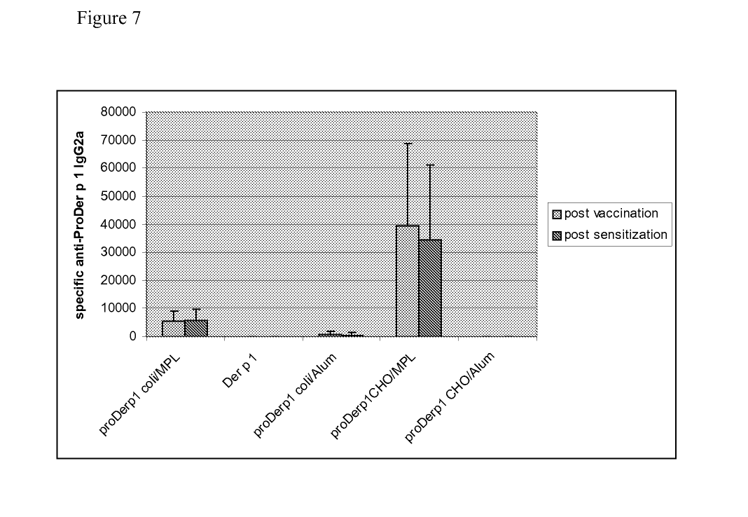

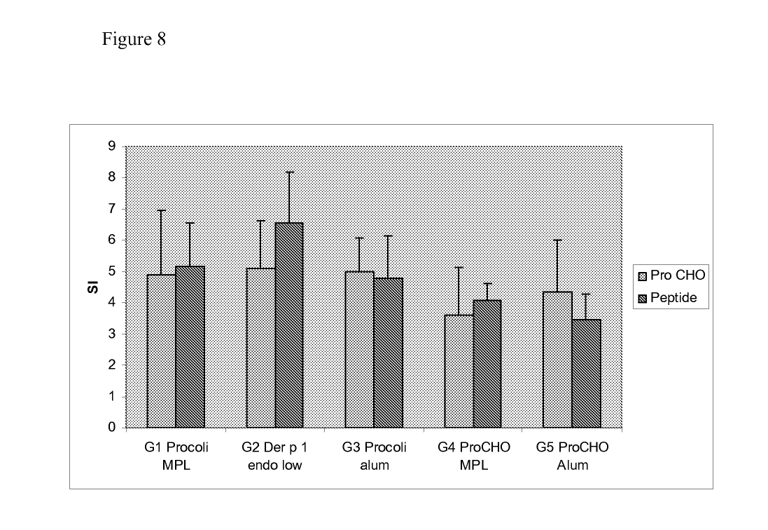

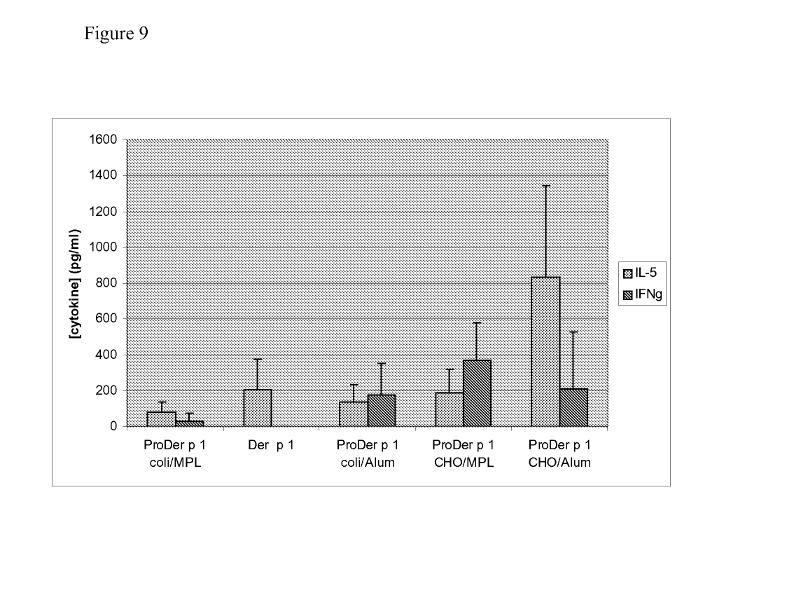

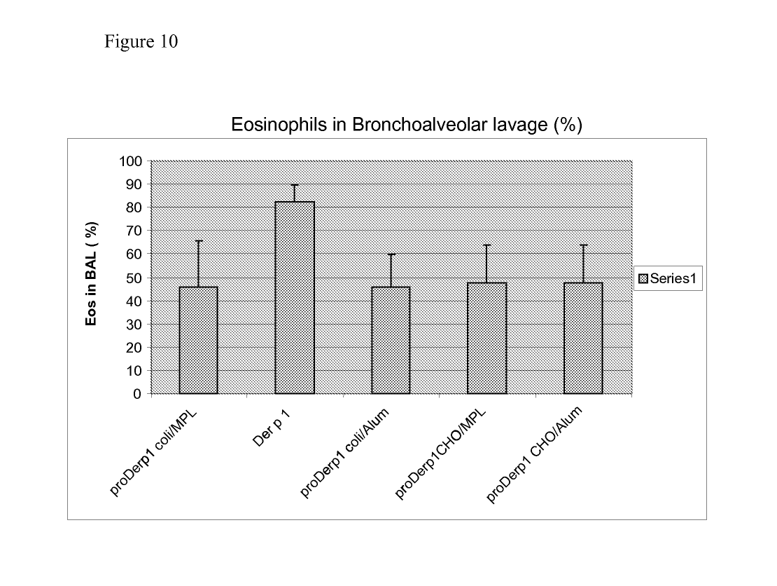

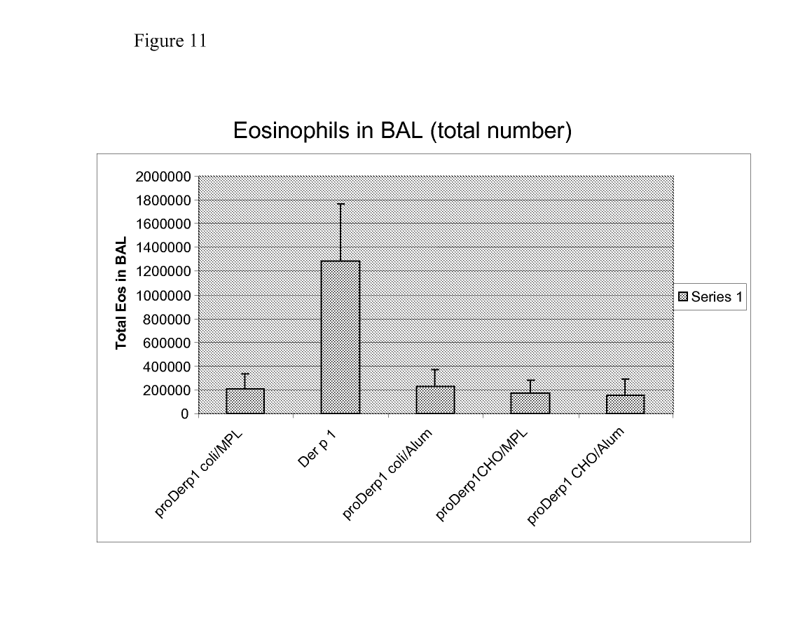

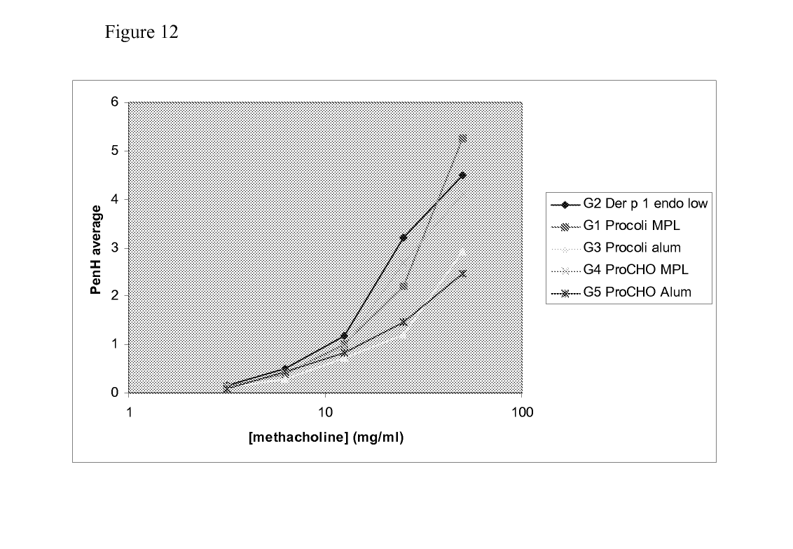

As shown in FIGS. 1A, B, and C, the IgE reactivity of the recombinant E. coli ProDer p 1 was drastically reduced compared to Der p 1 and ProDer p1 produced in mammalian cells. FIG. 2 shows the results of the Der p1 ELISA, confirming the absence of conformational epitopes in the allergen variant produced in the bacteria. However, the ProDer p 1 isolated from inclusion bodies maintained the Der p 1-specific T cell reactivity, as shown in FIG. 3.

SEQ ID NO: 1:
MASRP SSIKTFEEYK KAFNKSYATF EDEEAARKNF LESVKYVQSN
GGAINHLSDL SLDEFKNRFL MSAEAFEHLK TQFDLNAETN
ACSINGNAPA EIDLRQMRTV TPIRMQGGCG SCWAFSGVAA
TESAYLAYRN QSLDLAEQEL VDCASQHGCH GDTIPRGIEY
IQHNGVVQES YYRYVAREQS CRRPNAQRFG ISNYCQIYPP
NVNKIREALA QTHSAIAVII GIKDLDAFRH YDGRTIIQRD
NGYQPNYHAV NIVGYSNAQG VDYWIVRNSW DTNWGDNGYG
YFAANIDLMM IEEYPYVVIL RRREL

SEQ ID NO: 2:
ATGGCTAGCCGGCCGAGCTCCATTAAGACCTTCGAGGAATACAAGAAAGC
CTTCAACAAGAGCTATGCCACCTTCGAGGACGAGGAGGCCGCGCGCAAGA
ACTTCCTGGAAAGCGTGAAATACGTGCAGAGCAACGGCGGGGCTATAAAT
CACCTGTCCGACCTGTCTTTAGACGAGTTCAAGAACCGGTTCCTGATGAG
CGCCGAGGCTTTCGAACACCTTAAGACCCAGTTTGATCTCAACGCGGAGA
CCAACGCCTGCAGTATCAACGGCAATGCCCCCGCTGAGATTGATCTGCGC
CAGATGAGGACCGTGACTCCCATCCGCATGCAAGGCGGCTGCGGGTCTTG
TTGGGCCTTTTCAGGCGTGGCCGCGACAGAGTCGGCATACCTCGCGTATC
GGAATCAGAGCCTGGACCTCGCTGAGCAGGAGCTCGTTGACTGCGCCTCC
CAACACGGATGTCATGGGGATACGATTCCCAGAGGTATCGAATACATCCA
GCATAATGGCGTCGTGCAGGAAAGCTATTACCGATACGTAGCTAGGGAGC
AGTCCTGCCGCCGTCCTAACGCACAGCGCTTCGGCATTTCCAATTATTGC
CAGATCTACCCCCCTAATGCCAACAAGATCAGGGAGGCCCTGGCGCAGAC
GCACAGCGCCATCGCTGTCATCATCGGAATCAAGGATCTGGACGCATTCC
GGCACTATGACGGGCGCACAATCATCCAGCGCGACAACGGATATCAGCCA
AACTACCACGCGGTCAACATCGTGGGTTACTCGAACGCCCAGGGGGTGGA
CTACTGGATCGTGAGAAACAGTTGGGACACTAACTGGGGCGACAACGGCT
ACGGCTACTTCGCCGCCAACATCGACCTGATGATGATCGAGGAGTACCCG
TACGTGGTGATCCTGGCGGCCGCACTCGAGTGA

| SEQ ID NO: 3 (nucleotide sequence): | |
|---|---|
| SEQ ID NO: 7 (amino acid sequence): | |
| cgg ccg agc tcc att aag acc ttc gag gaa tac aag aaa gcc ttc aac | 48 |
| Arg Pro Ser Ser Ile Lys Thr Phe Glu Glu Tyr Lys Lys Ala Phe Asn | |
| 1 5 10 15 | |
| aag agc tat gcc acc ttc gag gac gag gag gcc gcg cgc aag aac ttc | 96 |
| Lys Ser Tyr Ala Thr Phe Glu Asp Glu Glu Ala Ala Arg Lys Asn Phe | |
| 20 25 30 | |
| ctg gaa agc gtg aaa tac gtg cag agc aac ggc ggg gct ata aat cac | 144 |
| Leu Glu Ser Val Lys Tyr Val Gln Ser Asn Gly Gly Ala Ile Asn His | |
| 35 40 45 | |
| ctg tcc gac ctg tct tta gac gag ttc aag aac cgg ttc ctg atg agc | 192 |
| Leu Ser Asp Leu Ser Leu Asp Glu Phe Lys Asn Arg Phe Leu Met Ser | |
| 50 55 60 | |
| gcc gag gct ttc gaa cac ctt aag acc cag ttt gat ctc aac gcg gag | 240 |
| Ala Glu Ala Phe Glu His Leu Lys Thr Gln Phe Asp Leu Asn Ala Glu | |
| 65 70 75 80 | |
| acc aac gcc tgc agt atc aac ggc aat gcc ccc gct gag att gat ctg | 288 |
| Thr Asn Ala Cys Ser Ile Asn Gly Asn Ala Pro Ala Glu Ile Asp Leu | |
| 85 90 95 | |
| cgc cag atg agg acc gtg act ccc atc cgc atg caa ggc ggc tgc ggg | 336 |
| Arg Gln Met Arg Thr Val Thr Pro Ile Arg Met Gln Gly Gly Cys Gly | |
| 100 105 110 | |
| tct tgt tgg gcc ttt tca ggc gtg gcc gcg aca gag tcg gca tac ctc | 384 |
| Ser Cys Trp Ala Phe Ser Gly Val Ala Ala Thr Glu Ser Ala Tyr Leu | |
| 115 120 125 | |
| gcg tat cgg aat cag agc ctg gac ctc gct gag cag gag ctc gtt gac | 432 |
| Ala Tyr Arg Asn Gln Ser Leu Asp Leu Ala Glu Gln Glu Leu Val Asp | |
| 130 135 140 | |
| tgc gcc tcc caa cac gga tgt cat ggg gat acg att ccc aga ggt atc | 480 |
| Cys Ala Ser Gln His Gly Cys His Gly Asp Thr Ile Pro Arg Gly Ile | |
| 145 150 155 160 | |
| gaa tac atc cag cat aat ggc gtc gtg cag gaa agc tat tac cga tac | 528 |
| Glu Tyr Ile Gln His Asn Gly Val Val Gln Glu Ser Tyr Tyr Arg Tyr | |
| 165 170 175 | |
| gta gct agg gag cag tcc tgc cgc cgt cct aac gca cag cgc ttc ggc | 576 |
| Val Ala Arg Glu Gln Ser Cys Arg Arg Pro Asn Ala Gln Arg Phe Gly | |
| 180 185 190 | |
| att tcc aat tat tgc cag atc tac ccc cct aat gcc aac aag atc agg | 624 |
| Ile Ser Asn Tyr Cys Gln Ile Tyr Pro Pro Asn Ala Asn Lys Ile Arg | |
| 195 200 205 | |
| gag gcc ctg gcg cag acg cac agc gcc atc gct gtc atc atc gga atc | 672 |
| Glu Ala Leu Ala Gln Thr His Ser Ala Ile Ala Val Ile Ile Gly Ile | |
| 210 215 220 | |
| aag gat ctg gac gca ttc cgg cac tat gac ggg cgc aca atc atc cag | 720 |
| Lys Asp Leu Asp Ala Phe Arg His Tyr Asp Gly Arg Thr Ile Ile Gln | |
| 225 230 235 240 | |
| cgc gac aac gga tat cag cca aac tac cac gcg gtc aac atc gtg ggt | 768 |
| Arg Asp Asn Gly Tyr Gln Pro Asn Tyr His Ala Val Asn Ile Val Gly | |
| 245 250 255 | |
| tac tcg aac gcc cag ggg gtg gac tac tgg atc gtg aga aac agt tgg | 816 |
| Tyr Ser Asn Ala Gln Gly Val Asp Tyr Trp Ile Val Arg Asn Ser Trp | |
| 260 265 270 | |
| gac act aac tgg ggc gac aac ggc tac ggc tac ttc gcc gcc aac atc | 864 |
| Asp Thr Asn Trp Gly Asp Asn Gly Tyr Gly Tyr Phe Ala Ala Asn Ile | |
| 275 280 285 | |
| gac ctg atg atg atc gag gag tac ccg tac gtg gtg atc ctg | 906 |
| Asp Leu Met Met Ile Glu Glu Tyr Pro Tyr Val Val Ile Leu | |
| 290 295 300 | |
| taa | 909 |

All patent applications to which priority is claimed are incorporated by reference herein in their entirety. This invention is not to be limited in scope by the specific embodiments described herein. Indeed, various modifications of the invention in addition to those described herein will become apparent to those skilled in the art from the foregoing description. Such modifications are intended to fall within the scope of the appended claims. The disclosures of the patents, patent applications and publications cited herein are incorporated by reference in their entireties.

What is claimed is:

1. A method for making a recombinant allergen with reduced IgE binding as compared to native allergen comprising the steps of (i) expressing said recombinant allergen in a host cell, and (ii) selecting said recombinant allergen having an increased IgE reducing structure as compared to said recombinant allergen expressed in a wild-type P. pastoris cell or a wild-type CHO cell.

2. The method according to claim 1, wherein the host cell is selected from the group consisting of: a prokaryotic host cell, an E. coli, a modified CHO cell, and a modified P. pastoris cell.

3. The method according to claim 1, wherein the host cell is a prokaryotic host cell.

4. The method according to claim 1, wherein the host cell is an E. coli cell.

5. The method according to claim 1, wherein the recombinant allergen is an allergen with proteolytic activity.

6. The method according to claim 1, wherein the recombinant allergen is ProDer p 1.

7. The method according to claim 1, wherein the recombinant allergen is Der p 1.

8. The method according to claim 1, wherein the IgE reducing structure is selected from the group consisting of: an altered higher order structure, increased β-sheet content, and aggregation.

9. A recombinant allergen with reduced IgE binding as compared to native allergen, which is obtainable by the method according to claim 1.

10. An immunogenic composition comprising a recombinant allergen further comprising an increased IgE reducing structure as compared to said recombinant allergen expressed in a wild-type P. pastoris cell or wild-type CHO cell.

11. An immunogenic composition comprising a recombinant allergen with reduced IgE binding as compared to native allergen according to claim 10 and an adjuvant.

12. The immunogenic composition according to claim 11, wherein the adjuvant is a preferential stimulator of Th1-type immune responses.

13. The immunogenic composition according to claim 11, wherein the adjuvant comprises at least one selected from the group of: 3D-MPL, QS21, a CpG oligonucleotide, a polyethylene ether and a polyethylene ester.

14. The immunogenic composition according to claim 10, wherein the recombinant allergen is presented in an oil in water or a water in oil emulsion vehicle.

15. A method of treating a patient suffering from allergic responses, comprising administering to said individual a recombinant allergen according to claim 9.

16. A method of treating a patient suffering from allergic responses, comprising administering to said individual an immunogenic composition according to claim 10.

17. A method of preventing a patient susceptible to allergic responses, comprising administering to said individual a recombinant protein according to claim 9.

18. A method of preventing a patient susceptible to allergic responses, comprising administering to said individual an immunogenic composition according to claim 10.
